(19) United States
(12) Patent Application Publication
(10) Pub. No.: US 2003/0004116 A1
(43) Pub. Date: Jan. 2, 2003

(54) Noni extract for prevention of disease

(76) Inventors: Gheetha Ghai, Murray Hill, NJ (US); Chi-Tang Ho, East Brunswick, NJ (US); Robert T. Rosen, Monroe Township, NJ (US); Mingfu Wang, Piscataway, NJ (US); Charles D. Boyd, Honolulu, HI (US); Katalin Csiszar, Honolulu, HI (US)

Correspondence Address: William J. McNichol, Jr., 2500 One Liberty Place, 1650 Market Street, Philadelphia, PA, 19103, US

(21) Appl. No.: 10/150,213

(22) Filed: May 17, 2002

Related U.S. Application Data
(60) Provisional application No. 60/292,061, filed on May 18, 2001.

(51) Int. Cl. A61K 31/7012; A61K 35/78

(52) U.S. Cl. 514/25; 424/765

(57) ABSTRACT

Compositions are provided which are derived from extracting the pulp of Noni fruit with butanol. The butanol extract of Noni fruit contains active glycoside compounds which are useful in the prevention and treatment of disease.

This application claims priority to application 60/292,061 filed May 18, 2001, and the text of application Ser. No. 60/292,061 is incorporated by reference in its entirety herewith.

### BACKGROUND OF THE INVENTION

The native Hawaiians used many plants for treating illnesses and more than 180 species of endemic, indigenous and Polynesian-introduced plants were used by Hawaiian traditional medical practitioners. One of the most effective and commonly used plants was Noni, also known as the Indian mulberry. Noni belongs to the Rubiaceae family which is represented by 450 genera and 5,500 species and is mainly found in tropical areas of Africa, Asia and throughout Polynesia. The extracts of the fruit, leaves and bark were utilized to treat a wide variety of illnesses, including hypertension, asthma, diabetes, arthritis, menstrual cramps, diarrhea and various disorders of the urinary and respiratory tract (Abbott, I. A. 1992. La'au Hawai'i: Traditional Hawaiian Uses of Plants. Bishop Museum Press: Honolulu, Hi.). Non-prescription herbal preparations of Noni are readily available and claim to aid in the treatment of several diseases including cancer (Elkins, R. M. H. 1997. Noni: The Prize Herb of the South Pacific. Woodland Publishing: Pleasant Grove, Utah.).

Several studies have examined the physiological effects of Noni and its extracts. Damnacathal, an anthraquinone compound isolated from a chloroform extract of the root of Morinda citrifolia induced normal morphology and cytoskeletal structure in K-ras-NRK cells (Hiramatsu, T. et al. 1993. Cancer Lett. 73:161-166). This effect was reversible when the compound was withdrawn and appeared to be ras-specific as the compound had no effect on RSV-NRK cells expressing the src oncogene. In another study, a precipitate of an ethanol extract of the juice of the Noni fruit was shown to have anti-tumor activity (Hirazumi, A. et al. 1994. Proc. West. Pharmacol. 37:145-146). Syngeneic mice with intraperitoneally implanted Lewis lung carcinoma had an increased life span when administered the Noni extract, an effect that was blocked by co-administration of 2-chloroadenosine, a macrophage inhibitor, and cyclosporine, a T-cell inhibitor. Later work by the same group further characterized the immunomodulatory activity of the ethanol extract of Noni (Hirazumi, A. et al. 1996. Proc. West. Pharmacol. 39:25-27). Noni extract was shown to stimulate peripheral mononuclear cells to produce the cytokines IL-1β and TNF-α, important mediators of tumor cytostasis and cytotoxicity. In addition, there has been a report of the use of Noni by a Polynesian community for the treatment of breast cancer (Singh, Y. N. et al. 1984. J. Ethnopharmacology 12:305-329).

FR 2783137 teaches use of an extract of Noni fruit for use as a health-improving drink or dietary supplement. JP 8217686 discloses use of an extract of the dried roots of Noni plant to treat recurring infection of the upper respiratory tract caused by Helicobacter pylori. In another Japanese application, the extract of roots of the Noni plant are listed as useful for treatment of cancer of the stomach and liver; the extract is identified as containing nordamnacanthal and damnacanthal (JP 8208461). JP 6087737 describes use of an extract of the roots of Noni as an inhibitor of proliferation of HIV; the active agent is identified as 1-methoxy-2-formyl-3-hydroxyanthraquinone. U.S. Pat. No. 5,288,491 describes a process for preparing a palatable Noni fruit powder that can be used to treat a variety of disorders including diabetes, heart trouble, high blood pressure, kidney and bladder disorders, diarrhea, menstrual problems, fever, aches, and pains. FR 2673639 discloses use of fatty acid rich fractions of the Noni plant as an insecticide. WO 88053043 describes use of plant extracts from Noni for treatment of hepatitis B and AIDS. JP 62132829 also discloses an extract of Noni as useful for treatment of hepatitis, specifically naming asperulosidic acid as the extract active ingredient.

### SUMMARY OF THE INVENTION

An object of the present invention is a composition which comprises a butanol extract of Noni fruit pulp. The active compounds identified in this extract include 6-O-(β-D-glucopyranosyl)-1-O-octanoyl-β-D-glucopyranose (compound 1), 6-O-(β-D-glucopyranosyl)-1-O-hexanoyl-β-D-glucopyranose (compound 2), and 3-methylbut-3-enyl 6-O-β-D-glucopyranosyl-β-D-glucopyranoside (compound 3).

Another object of the present invention is a method for inhibiting tumor cell growth in an animal comprising administering to an animal a composition which comprises the butanol extract of Noni fruit.

Yet another object of the present invention is a method for preventing or treating cancer in an animal comprising administering to an animal an effective amount a composition which comprises the butanol extract of Noni fruit.

### DETAILED DESCRIPTION OF THE INVENTION

A butanol extract of Noni (Morinda citrifolia) fruit has been identified that contains 6-O-(β-D-glucopyranosyl)-1-O-octanoyl-β-D-glucopyranose (“compound 1”), 6-O-(β-D-glucopyranosyl)-1-O-hexanoyl-β-D-glucopyranose (“compound 2”), and 3-methylbut-3-enyl 6-O-β-D-glucopyranosyl-β-D-glucopyranoside (“compound 3”). This extract from Noni is useful in the prevention and treatment of cancer.

The first experiments involved extraction of Noni fruit with a water-based solution. Ripe Noni fruit was blended in a small amount of water and the seeds were removed by colander straining. After seed removal, 10 g of pulp was dissolved in 20 ml phosphate buffered saline (PBS). The mixture was mixed vigorously for 5 to 10 minutes then centrifuged for 10 minutes at 2500 rpm. The supernatant was recentrifuged for 10 minutes at 14,000 rpm. The resulting supernatant was collected and then tested for activity in cell proliferation assays and cytotoxicity studies.

Human breast carcinoma cells (MCF-7) and normal human fibroblast cells (HT-1080) were grown in culture. Cells in culture were treated with 50 μl of PBS-extracted Noni supernatant. Cell growth of the MCF-7 cells treated with the Noni extract was decreased by about 50% on day 3 and 80% on day 5. There were no significant changes in cell growth in the untreated MCF-7 cells which did not receive Noni extract. HT-1080 cells treated with Noni extract exhibited a 10% decrease in cell growth on day 3, a change that was not statistically significant when compared to untreated cells. In addition, the cell growth had returned to normal levels by day 5. When examined with light microscopy, MCF-7 cells treated with extract were observed to have cytoskeletal changes. While untreated MCF-7 cells appeared round, flat and clumped, treated MCF-7 cells were stretched in appearance with cytoskeletal structures similar to normal human fibroblasts and did not grow in typical clumps. There were no phenotypic changes in the HT-1080 cells treated with Noni extract.

Cytotoxicity testing was performed in HCT-116 cells grown in culture. Noni extract was added to cells at concentrations of 0, 25, 50, 100, 250, 500 and 1000 mg/ml and incubated for 24 hours. Cytotoxicity was seen at extract concentrations of 100 to 250 mg/ml.

In addition to testing of the PBS-extracted Noni supernatant, Noni fruit was freeze-dried and lyophilized for fractionation. Four fractions with increasing polarity were obtained from an original 200 g sample. These were a hexane fraction (0.4 g), an ethyl acetate fraction (4 g), a butanol fraction (45 g), and a water fraction (60 g).

The four extracts of Noni fruit were also evaluated in the cytotoxicity assay to determine which fractions contained active compounds. This testing was performed in the breast cell line (MCF-7), the colon carcinoma cell line (HCT-116) and in the prostate carcinoma cell line (DU-145). The butanol fraction tested contained the three novel glycoside compounds, compounds 1, 2 and 3. The concentrations of each of the four extracts were administered in proportion to the mount of each fraction isolated from the pulp of the Noni fruit and were also chosen based on the results of the cytotoxicity testing of the fresh PBS-extracted Noni fruit. Therefore, the concentrations evaluated for the water extract were 0, 3, 9, 15, 22 and 30 mg/ml. The concentrations for the butanol extract tested were approximately 66% of those used for the water extract. Ethyl acetate and hexane extracts were tested at concentrations that were 10% and 1%, respectively, of the butanol extract levels. Two sets of assays were performed. Both sets involved incubation of each of the carcinoma cell lines with each of the four Noni extracts for at least 24 hours. For one set, the media with the Noni extracts were removed after the designated treatment period and replaced with fresh media in order to evaluate the ability of the cells to recover in the absence of Noni extracts. Assays were performed at the end of the initial 24 treatment period and after a 72 hours recovery period. The exception was the ethyl acetate-extracted Noni treated cells which were evaluated after 48 hours of recovery following 48 hours of treatment and the hexane extract treated cells which were evaluated after 96 hours of treatment. The results showed that after 24 hours of treatment, the water extract of Noni was cytotoxic at a dose of approximately 9 mg/ml. The butanol extract of Noni was cytotoxic at a dose of about 6 mg/ml in the HCT-116 and DU-145 cells while the MCF-7 cells required higher doses of about 10 mg/ml. The ethyl acetate extract of Noni was cytotoxic at a dose of about 1.4 mg/ml in DU-145 and MCF-7 cells, while HCT-116 cells required about 2.0 mg/ml of the extract for a similar cytotoxic effect. The hexane extract of Noni showed no cytotoxicity, even when the treatment was extended for 96 hours and the cells were treated with a dose 10 times higher than the original treatment level.

The water, butanol and ethyl acetate fractions of Noni all showed cytotoxic activity following 24 hours of treatment. However, the cells were allowed to recover in the absence of the fractions, with some cell lines showing a high level of proliferative activity at recovery. Although the water fraction was cytotoxic at 9 mg/ml after 24 hours of treatment, after 72 hours in the absence of the fraction, only those cells treated with concentrations of 15 mg /ml and higher were unable to recover. DU-145 cells were able to recover when treated with concentrations less than 30 mg/ml. HCT-116 and MCF-7 cells treated with the butanol fraction were unable to recover, although DU-145 cells recovered at all doses tested. All cells treated with the ethyl acetate fraction recovered even after extended treatments of 48 hours . These data indicate that the butanol fraction had the most effective compounds for activity as anti-proliferative agents and anti-tumor agents.

In the butanol soluble fraction of Noni fruit extract, three novel glycoside compounds were identified. Compound 1 was obtained as a white powder. The negative APCI-MS exhibited a pseudomolecular ion peak at m/z 467 [M−1]⁻ and the positive APCI-MS showed a significant pseudomolecular ion peak at m/z 486 [M+NH₄]⁺, compatible with the molecular formula C₂₀H₃₆O₁₂. In the ¹H NMR and ¹³C NMR, compound 1 showed signals consistent with an octanoyl partial structure. In the ¹H NMR spectrum of compound 1, two anomeric proton signals at δ5.45(1H, d, J=7.8 Hz) and 4.31(1H, d, J=7.8 Hz) were observed. The ¹³C NMR also displayed signals at δ104.5(d), 77.9(d), 77.9(d), 75.0(d), 71.4(d), and 62.6(t), attributable to terminal β-D-glucose, and signals at δ95.5(d), 77.7(d), 77.7(d), 73.8(d), 70.8(d), and 69.4(t) for the inner glucose. Comparison with literature values indicated 1-6 linkage of these two glucose units and the octanoyl moiety was placed on the anomeric carbon of the central glucose. The above evidence established the structure of compound 1 as 6-O-(β-D-glucopyranosyl)-1-O-octanoyl-β-D-glucopyranose.

The structure of compound 1was confirmed by ¹H-¹H COSY, NOESY, HMQC and HMBC spectra. HMBC experiments showed correlation contours between H-1 of the central glucose (δ5.45) and the carbonyl carbon of the octanoyl moiety (δ174.1), and between H-1 of the terminal glucose (δ4.31) and C-6 of the central glucose (δ69.4).

Compound 2 was also obtained as a white powder. The negative APCI-MS exhibited a significant pseudomolecular ion peak at m/z 439 [M−1]⁻ and the positive APCI-MS showed an ion peak at m/z 458 [M+NH₄]⁺. These MS data together with the ¹H NMR and ¹³C NMR data suggested the molecular formula C₁₈H₃₂O₁₂. The IR spectrum showed hydroxyl and carbonyl absorptions. In the ¹H NMR spectrum, compound 2 showed signals similar to those of compound 1. Only slight differences were observed in the high field where instead of signals for an octanoyl moiety, signals for a hexanoyl moiety were observed. This was further supported by the ¹³C NMR spectrum which showed signals at δ14.3(q), 23.4(t), 25.3(t), 32.3(t), 34.8(t) and 174.1(s), assignable to a hexanoyl moiety. The remaining ¹³C NMR signals for the two glucose moieties were identical with those of compound 1. The ¹H NMR signals for the two anomeric protons were observed at δ5.45 and 4.31. Analysis of the ¹H-¹H COSY, HMQC and HMBC spectra led to assignment of all ¹H NMR and ¹³C NMR signals for compound 2. Thus, compound 2 as identified to be 6-O-(β-D-glucopyranosyl)-1-O-hexanoyl-β-D-glucopyranose.

Compound 3 exhibited a significant pseudomolecular ion peak at m/z 409 [M−1]⁻ in negative APCI-MS and an ion peak at 428 [M+NH₄]⁺ in the positive APCI-MS. MS data together with the ¹H NMR and ¹³C NMR data suggested molecular formula C₁₇H₃₀O₁₁. The ¹H NMR and ¹³C NMR spectra of compound 3 showed the signals for a 1-6 linked β-D-glucopyranoysl-β-D-glucopyranose moiety. In addition to signals for sugars, the ¹H NMR spectrum showed the presence of one methyl (δ1.75), two methylenes [δ2.35(2H), 3.65 (1H), 3.99(1H)] and one exomethylene [δ4.74(1H) and 4.75 (1H)], while the remaining ¹³C signals were observed at δ23.0(q), 38.7(t), 69.5(t), 112.1(t) and 143.9(s). These data were assignable to the partial structure CH₂═C(CH₃)CH₂CH₂O—.

Analysis of the ¹H-¹H COSY, HMQC and HMBC spectra led to assignment of the ¹H NMR and ¹³C NMR data of compound 3 and confirmed the partial structure moiety of CH₂═C(CH₃)CH₂CH₂O—. A ¹³C signal at 112.1(t) in the HMQC spectrum correlated with the exomethylene proton signals (δ4.74 and 4.75). The former signal also showed correlation with carbon signals at δ143.9(C-3) and 38.7(C-2), while the latter signal showed correlation with carbon signals at δ143.9 and 23.0(C-5) in the HMBC spectrum. The linkage of sugars and aglycone was consistent with the HMBC experiments, in which correlations were observed between H-1 of the central glucose (δ4.27) and a CH₂ at δ69.5. Thus, the structure of compound 3 was elucidated as 3-methylbut-3-enyl 6-O-β-D-glucopyranoysl-β-D-glucopyranoside.

These three glycosides from the butanol fraction of Noni fruit, which can also be synthesized, are compounds with potential activity as anti-tumor and anti-proliferative agents. Thus, the present invention provides compositions having 6-O-(β-D-glucopyranosyl)-1-O-octanoyl-β-D-glucopyranose, 6-O-(β-D-glucopyranosyl)-1-O-hexanoyl-β-D-glucopyranose and 3-methylbut-3-enyl 6-O-β-D-glucopyranosyl-β-D-glucopyranoside. The present invention provides also methods for inhibiting growth of tumor cells with the above compositions. Also, the data provided herein for Noni fruit extracts, specifically the butanol extract and its three identified compounds, support the development of foods and dietary supplements having the three glycoside compounds for animal consumption. For purposes of the present invention by “animal” it is meant to include humans. These foods and supplements are referred to by those of skill in the art as “nutraceuticals”. Based upon the experiments described herein, it is expected that comopounds 1, 2 and 3 can be synthesized and compositions having compounds 1, 2 and 3 of the present invention can be used as nutraceuticals for prevention or treatment of cancer. One of skill can use the results of experiments in cells and animals described herein to determine effective amounts to be administered to other animals, including humans. By “effective amount” it is meant a concentration that inhibits tumor growth either in vitro in cells or in vivo in animals. For example, human test doses can be extrapolated from effective doses in cell studies, such as IC₅₀ values, or from effective doses in vivo by extrapolating on a body weight or surface area basis. Such extrapolations are routine in the art.

Compositions comprising compounds 1, 2 and 3 can be formulated for administration as a food supplement using one or more fillers. Alternatively, compositions comprising these extracts can be administered as conventional pharmaceuticals using one or more physiologically acceptable carriers or excipients. Nutraceutical compositions can be formulated for administration by any route including, but not limited to, inhalation or insufflation (through mouth or nose), oral, buccal, parenteral, vaginal, or rectal administration. In one embodiment, oral administration, the compositions are added directly to foods and ingested as part of a normal meal. Various methods are known to those skilled in the art for addition or incorporation of nutraceuticals into foods.

Compositions for use in the present invention can also be administered in the form or tablets or capsules prepared by conventional means with pharmaceutically acceptable excipients such as binding agents, fillers, lubricants, disintegrants, or wetting agents. Examples of specific compounds for use in formulating tablets and capsules are described in detail in the U.S. Pharmacopeia. Tablets comprising the extract can also be coated by methods well known in the art. Liquid preparations for oral administration can also be used. Liquid preparations can be in the form of solutions, syrups or suspensions, or a dry product for reconstitution with water or another suitable vehicle before use. Such liquid preparations can be prepared by conventional means with pharmaceutically acceptable additives such as suspending agents, emulsifying agents, non-aqueous vehicles, and preservatives. Again, specific additives are well known to those of skill and are listed in places such as the U.S. Pharmacopeia. In one embodiment, the oral preparation is formulated to provide controlled time release of the active nutraceutical components. For buccal administration the extract can be formulated as a tablet or lozenge.

For administration by inhalation, compositions for use in the present invention can be delivered in the form of an aerosol spray in a pressurized package or as a nebulizer, with use of suitable propellants. In the case of a pressurized aerosol, the dosage unit can be determined by providing a valve to deliver a metered dose.

Parenterally administered compositions are formulated to allow for injection, either as a bolus or as a continuous infusion. Formulations for injection can be prepared in unit dosage forms, such as ampules, or in multi-dose units, with added preservatives. The compositions for injection can be in the form of suspensions, solutions, or emulsions, in either oily or aqueous vehicles. They may also contain formulatory agents such as suspending agents, stabilizing agents, and/or dispersing agents. The active ingredient may also be presented in powder form for reconstitution with a suitable vehicle before use. Specific examples of formulating agents for parenteral injection are found in the U.S. Pharmacopeia.

For rectal administration or vaginal administration, compositions for use in of the present invention can be formulated as suppositories, creams, gels, or retention enemas.

For dietary supplements, the extract can be added in concentrations up to 5% by weight and mixed according to methods routine in the art. Dietary supplements for animals can be prepared in a variety of forms including, but not limited to, liquid, powder, or solid pill forms. In the present invention, compounds 1, 2 and 3 can administered either alone or in combination with other phytochemicals known to affect tumor cell growth, where combining compounds or extracts would lead to synergistic effects.

What is claimed is:

1. A composition comprising a butanol extract of Noni fruit pulp.

2. The composition of claim 1 wherein said butanol extract comprises 6-O-(β-D-glucopyranosyl)-1-O-octanoyl-β-D-glucopyranose, 6-O-(β-D-glucopyranosyl)-1-O-hexanoyl-β-D-glucopyranose, and 3-methylbut-3-enyl 6-O-β-D-glucopyranosyl-β-D-glucopyranoside.

3. A method for inhibiting tumor cell growth in an animal comprising administering to an animal the composition of claim 1.

4. A method for preventing or treating cancer in an animal comprising administering to an animal an effective amount of the composition of claim 1.

5. A composition comprising 6-O-(β-D-glucopyranosyl)-1-O-octanoyl-β-D-glucopyranose, 6-O-(β-D-glucopyranosyl)-1-O-hexanoyl-β-D-glucopyranose, and 3-methylbut-3-enyl 6-O-β-D-glucopyranosyl-β-D-glucopyranoside.

6. A method for inhibiting growth of tumor cells comprising administering to said cells the composition of claim 5.

7. A method for preventing or treating cancer in an animal comprising administering to an animal an effective amount of the composition of claim 5.
